(19) United States
(12) Patent Application Publication
(10) Pub. No.: US 2012/0213862 A1
(43) Pub. Date: Aug. 23, 2012

(54) Negative Ion-Releasing Silica Compositions and Methods of Preparing and Using the Same

(75) Inventor: John Clark Walton, Fleming Island, FL (US)

(73) Assignee: ENERGY BALANCE GEAR LLC, Orange Park, FL (US)

(21) Appl. No.: 13/102,692

(22) Filed: May 6, 2011

Related U.S. Application Data
(60) Provisional application No. 61/444,938, filed on Feb. 21, 2011.

(51) Int. Cl. A61K 33/24; A61P 3/00; A61P 43/00; A61P 37/08; A61K 33/26; A61K 33/42

(52) U.S. Cl. 424/604; 424/617; 424/646

(57) ABSTRACT

Negative ion-releasing silica compositions and methods for their preparation and use are described herein. The compositions include silicon dioxide and gadolinium. Further described herein are products including the compositions.

### CROSS-REFERENCE TO PRIORITY APPLICATION

This application claims priority to U.S. Provisional Application No. 61/444,938, filed Feb. 21, 2011, which is incorporated herein by reference in its entirety.

### BACKGROUND

Negative ions have several advantageous properties, including the ability to enhance health, reduce susceptibility to allergies, and aid in human metabolism. Living organisms are naturally exposed to negative ions in the environment, with significant amounts being present near waterfalls (e.g., about 50,000 ions per cc) and in mountainous regions (e.g., from about 5,000 ions per cc). However, negative ion levels are substantially lower in urban settings and within closed environments, where levels of negative ions exist at a mere 50 ions per cc.

### SUMMARY

Negative ion-releasing silica compositions and methods for their preparation and use are described herein. The compositions include silicon dioxide and gadolinium. In some examples, the silicon dioxide is present in an amount of 20% to 30% based on the weight of the composition. In some examples, the gadolinium is present in an amount of 0.1% to 0.5% based on the weight of the composition.

The compositions described herein can further include one or more additional components. For example, the compositions can further include a lanthanide (e.g., neodymium, praseodymium, cerium, or mixtures of these). In some examples, the lanthanide is present in an amount of 2% to 5% based on the weight of the composition. In some embodiments, the compositions described herein can further include tourmaline, germanium, zeolite powder, hematite, or a mixture of these. The compositions can further include an additional oxide (e.g., aluminum oxide, calcium oxide, iron oxide, magnesium oxide, sodium oxide, sulfur dioxide, titanium dioxide, zirconium dioxide, or mixtures of these). In some examples, the total amount of additional oxide is present in an amount of 40% to 65% based on the weight of the composition. Optionally, the compositions described herein further include a mineral acid (e.g., phosphoric acid).

Also described herein is a composition including silicon dioxide in an amount of 23% to 26% based on the weight of the composition; gadolinium in an amount of 0.1% to 0.3% based on the weight of the composition; aluminum oxide in an amount of 1.6% to 1.9% based on the weight of the composition; calcium oxide in an amount of 0.1% to 0.5% based on the weight of the composition; iron oxide in an amount of 0.1% to 0.5% based on the weight of the composition; sodium oxide in an amount of 0.01% to 0.1% based on the weight of the composition; titanium oxide in an amount of 0.2% to 0.4% based on the weight of the composition; zirconium dioxide in an amount of 50% to 60% based on the weight of the composition; phosphoric acid in an amount of 6% to 8% based on the weight of the composition; cerium in an amount of 0.1% to 1% based on the weight of the composition; and sulfur dioxide in an amount of 0.01% to 0.1% based on the weight of the composition. In some examples, the composition further includes neodymium in an amount of 1% to 2% based on the weight of the composition; and praseodymium in an amount of 1% to 2% based on the weight of the composition. In some examples, the composition further includes zeolite powder in an amount of 1% to 2% based on the weight of the composition; and tourmaline in an amount of 1% to 2% based on the weight of the composition.

Also provided are products including the compositions described herein. Examples of the products include wristbands, shoe insoles, clothing items (e.g., shirts), and hats. In some examples, the products described herein further include an adhesive composition that optionally includes tourmaline.

Methods of delivering the compositions described herein to a subject are also provided. The methods include applying the composition to a product and contacting a subject with the product. In some examples, the product is a clothing item (e.g., a shirt) or a wristband.

Further provided are methods of making the compositions described herein. The methods include mixing silicon dioxide, gadolinium, and optionally, any of the one or more components described herein.

The details of one or more embodiments are set forth in the description below. Other features, objects, and advantages will be apparent from the description and from the claims.

### DETAILED DESCRIPTION

Negative ion-releasing silica compositions and methods for their preparation and use are described herein. These compositions are suitable for releasing a quantity of negative ions into the environment. The compositions include silicon dioxide, gadolinium, and other negative ion releasing components. Optionally, the compositions include additional negative ion releasing components, such as metals.

The silicon dioxide (i.e., silica or SiO₂) for use in the methods described herein can be obtained from commercial sources, such as Sigma Aldrich (St. Louis, Mo.); Acros Organics (Morris Plains, N.J.); or Fisher Scientific (Hampton, N.H.). Suitable silicon dioxide grades include powders with mean particle sizes from about 2 nm to about 100 μm. For example, the mean particle size of the silicon dioxide can be about 2 nm, about 5 nm, about 10 nm, about 20 nm, about 50 nm, about 100 nm, about 500 nm, about 1 μm, about 5 μm, about 10 μm, about 20 μm, about 30 μm, about 40 μm, about 50 μm, about 60 μm, about 70 μm, about 80 μm, about 90 μm, or about 100 μm. For example, the silicon dioxide can include particle sizes from about 5 nm to about 15 nm, from 10 nm to 20 nm, or about 10 μm to about 20 μm.

Silicon dioxide can be included in the compositions described herein in an amount of about 10% to about 40% based on the weight of the composition. For example, the total amount of silica in the composition can be from about 12% to about 27%, from about 15% to about 35%, or from about 20% to about 30% based on the weight of the composition. In some examples, the total amount of silica in the composition is less than about 40%, less than about 35%, less than about 30%, or less than about 25% based on the weight of the composition.

As described above, the compositions described herein further include gadolinium. Gadolinium is a rare earth metal, classified elementally as a lanthanide, that can be obtained from minerals, such as gadolinite, monazite, and bastnasite. Gadolinium can be isolated by chemically treating the minerals and purifying the resulting products using extraction and chromatographic methods (e.g., ion exchange chromatography). The gadolinium can be introduced into the composition as elemental gadolinium or can be included in a composite material, (e.g., monazite or bastnasite).

The gadolinium can be included in the compositions described herein in an amount from about 0.005% to 1% based on the weight of the composition. In some examples, gadolinium can be included in the compositions in an amount from about 0.01% to about 1% (e.g., from about 0.1% to about 0.5%). For example, the total amount of gadolinium present can be greater than about 0.01%, greater than about 0.02%, greater than about 0.03%, greater than about 0.04%, greater than about 0.05%, greater than about 0.06%, greater than about 0.07%, greater than about 0.08%, greater than about 0.09%, greater than about 0.1%, greater than about 0.11%, greater than about 0.12%, greater than about 0.13%, greater than about 0.14%, greater than about 0.15%, greater than about 0.16%, greater than about 0.17%, greater than about 0.18%, greater than about 0.19%, greater than about 0.2%, greater than about 0.21%, greater than about 0.22%, greater than about 0.23%, greater than about 0.24%, greater than about 0.25%, greater than about 0.26%, greater than about 0.27%, greater than about 0.28%, greater than about 0.29%, greater than about 0.3%, greater than about 0.31%, greater than about 0.32%, greater than about 0.33%, greater than about 0.34%, greater than about 0.35%, greater than about 0.36%, greater than about 0.37%, greater than about 0.38%, greater than about 0.39%, greater than about 0.4%, greater than about 0.41%, greater than about 0.42%, greater than about 0.43%, greater than about 0.44%, greater than about 0.45%, greater than about 0.46%, greater than about 0.47%, greater than about 0.48%, or greater than about 0.49% based on the weight of the composition.

The compositions described herein can further include one or more additional lanthanides as negative ion releasing components. For example, the compositions can further include neodymium, praseodymium, cerium, or mixtures of these. The lanthanides can be introduced into the compositions in their elemental forms or can be included as a component of a composite material. For example, neodymium, praseodymium, and cerium can each be introduced as a component of a mineral (e.g., monazite or bastnasite). The one or more additional lanthanides can be present in an amount from about 1% to about 10% based on the weight of the composition (e.g., from about 2% to about 5% based on the weight of the composition). For example, the amount of lanthanides present, excluding gadolinium, can be greater than about 2.5%, greater than about 3%, greater than about 3.5%, greater than about 4%, or greater than about 4.5% based on the weight of the composition.

In some examples, neodymium can be included in the compositions in an amount from about 0.5% to about 2.5%. For example, the total amount of praseodymium present can be greater than about 0.6%, greater than about 0.7%, greater than about 0.8%, greater than about 0.9%, greater than about 1%, greater than about 1.1%, greater than about 1.2%, greater than about 1.3%, greater than about 1.4%, greater than about 1.5%, greater than about 1.6%, greater than about 1.7%, greater than about 1.8%, greater than about 1.9%, greater than about 2.0%, greater than about 2.1%, greater than about 2.2%, greater than about 2.3%, or greater than about 2.4% based on the weight of the composition.

In some examples, praseodymium can be included in the compositions in an amount ranging from about 0.5% to about 2%. For example, the total amount of praseodymium present can be greater than about 0.6%, greater than about 0.7%, greater than about 0.8%, greater than about 0.9%, greater than about 1%, greater than about 1.1%, greater than about 1.2%, greater than about 1.3%, greater than about 1.4%, greater than about 1.5%, greater than about 1.6%, greater than about 1.7%, greater than about 1.8%, or greater than about 1.9% based on the weight of the composition.

In some examples, cerium can be included in the compositions in an amount from about 0.1% to about 1%. For example, the total amount of cerium present can be greater than about 0.2%, greater than about 0.3%, greater than about 0.4%, greater than about 0.5%, greater than about 0.6%, greater than about 0.7%, greater than about 0.8%, or greater than about 0.9% based on the weight of the composition.

The compositions described herein can further include one or more additional oxides. In some examples, the one or more additional oxides can include one or more of aluminum oxide, calcium oxide, iron oxide, magnesium oxide, sodium oxide, sulfur dioxide, titanium dioxide, zirconium dioxide, and mixtures of these. The one or more additional oxides can be present in a total amount from about 40% to about 65% based on the weight of the composition. In some examples, aluminum oxide (i.e., alumina) can be present in an amount from about 0.5% to about 2.5% by weight of the composition. For example, aluminum oxide can be present in an amount of about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, or about 2.4% based on the weight of the composition.

Calcium oxide, iron oxide, magnesium oxide, and titanium dioxide (i.e., titania) can each optionally be present in amounts from about 0.01% to about 0.5% based on the weight of the composition. For example, the calcium oxide present can be greater than about 0.02%, greater than about 0.03%, greater than about 0.04%, greater than about 0.05%, greater than about 0.06%, greater than about 0.07%, greater than about 0.08%, greater than about 0.09%, greater than about 0.1%, greater than about 0.11%, greater than about 0.12%, greater than about 0.13%, greater than about 0.14%, greater than about 0.15%, greater than about 0.16%, greater than about 0.17%, greater than about 0.18%, greater than about 0.19%, greater than about 0.2%, greater than about 0.21%, greater than about 0.22%, greater than about 0.23%, greater than about 0.24%, greater than about 0.25%, greater than about 0.26%, greater than about 0.27%, greater than about 0.28%, greater than about 0.29%, greater than about 0.3%, greater than about 0.31%, greater than about 0.32%, greater than about 0.33%, greater than about 0.34%, greater than about 0.35%, greater than about 0.36%, greater than about 0.37%, greater than about 0.38%, greater than about 0.39%, greater than about 0.4%, greater than about 0.41%, greater than about 0.42%, greater than about 0.43%, greater than about 0.44%, greater than about 0.45%, greater than about 0.46%, greater than about 0.47%, greater than about 0.48%, or greater than about 0.49% based on the weight of the composition.

Additionally, iron oxide present can be greater than about 0.02%, greater than about 0.03%, greater than about 0.04%, greater than about 0.05%, greater than about 0.06%, greater than about 0.07%, greater than about 0.08%, greater than about 0.09%, greater than about 0.1%, greater than about 0.11%, greater than about 0.12%, greater than about 0.13%, greater than about 0.14%, greater than about 0.15%, greater than about 0.16%, greater than about 0.17%, greater than about 0.18%, greater than about 0.19%, greater than about 0.2%, greater than about 0.21%, greater than about 0.22%, greater than about 0.23%, greater than about 0.24%, greater than about 0.25%, greater than about 0.26%, greater than about 0.27%, greater than about 0.28%, greater than about 0.29%, greater than about 0.3%, greater than about 0.31%, greater than about 0.32%, greater than about 0.33%, greater than about 0.34%, greater than about 0.35%, greater than about 0.36%, greater than about 0.37%, greater than about 0.38%, greater than about 0.39%, greater than about 0.4%, greater than about 0.41%, greater than about 0.42%, greater than about 0.43%, greater than about 0.44%, greater than about 0.45%, greater than about 0.46%, greater than about 0.47%, greater than about 0.48%, or greater than about 0.49% based on the weight of the composition.

Also, magnesium oxide present can be greater than about 0.02%, greater than about 0.03%, greater than about 0.04%, greater than about 0.05%, greater than about 0.06%, greater than about 0.07%, greater than about 0.08%, greater than about 0.09%, greater than about 0.1%, greater than about 0.11%, greater than about 0.12%, greater than about 0.13%, greater than about 0.14%, greater than about 0.15%, greater than about 0.16%, greater than about 0.17%, greater than about 0.18%, greater than about 0.19%, greater than about 0.2%, greater than about 0.21%, greater than about 0.22%, greater than about 0.23%, greater than about 0.24%, greater than about 0.25%, greater than about 0.26%, greater than about 0.27%, greater than about 0.28%, greater than about 0.29%, greater than about 0.3%, greater than about 0.31%, greater than about 0.32%, greater than about 0.33%, greater than about 0.34%, greater than about 0.35%, greater than about 0.36%, greater than about 0.37%, greater than about 0.38%, greater than about 0.39%, greater than about 0.4%, greater than about 0.41%, greater than about 0.42%, greater than about 0.43%, greater than about 0.44%, greater than about 0.45%, greater than about 0.46%, greater than about 0.47%, greater than about 0.48%, or greater than about 0.49% based on the weight of the composition.

Further, the titanium dioxide present can be greater than about 0.02%, greater than about 0.03%, greater than about 0.04%, greater than about 0.05%, greater than about 0.06%, greater than about 0.07%, greater than about 0.08%, greater than about 0.09%, greater than about 0.1%, greater than about 0.11%, greater than about 0.12%, greater than about 0.13%, greater than about 0.14%, greater than about 0.15%, greater than about 0.16%, greater than about 0.17%, greater than about 0.18%, greater than about 0.19%, greater than about 0.2%, greater than about 0.21%, greater than about 0.22%, greater than about 0.23%, greater than about 0.24%, greater than about 0.25%, greater than about 0.26%, greater than about 0.27%, greater than about 0.28%, greater than about 0.29%, greater than about 0.3%, greater than about 0.31%, greater than about 0.32%, greater than about 0.33%, greater than about 0.34%, greater than about 0.35%, greater than about 0.36%, greater than about 0.37%, greater than about 0.38%, greater than about 0.39%, greater than about 0.4%, greater than about 0.41%, greater than about 0.42%, greater than about 0.43%, greater than about 0.44%, greater than about 0.45%, greater than about 0.46%, greater than about 0.47%, greater than about 0.48%, or greater than about 0.49% based on the weight of the composition.

Sodium oxide and sulfur dioxide can each be present in the composition in amounts ranging from about 0.01% to about 0.1%. For example, sodium oxide can be present in an amount of about 0.02%, about 0.03%, about 0.04%, about 0.05%, about 0.06%, about 0.07%, about 0.08%, or about 0.09% based on the weight of the composition. Sulfur dioxide can also be present in an amount of about 0.02%, about 0.03%, about 0.04%, about 0.05%, about 0.06%, about 0.07%, about 0.08%, or about 0.09% based on the weight of the composition.

In some embodiments, zirconium dioxide (i.e., zirconia) is present in the composition. Zirconium dioxide can be present in an amount ranging from about 40% to about 60% based on the weight of the composition. For example, zirconium dioxide can be present in an amount of greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 52%, greater than about 53%, greater than about 54%, greater than about 55%, greater than about 56%, greater than about 57%, greater than about 58%, or greater than about 59% based on the weight of the composition.

The compositions described herein can further include a mineral acid. Examples of suitable mineral acids include phosphoric acid, nitric acid, sulfuric acid, and boric acid. The mineral acid can be present in an amount ranging from about 3% to about 10% based on the weight of the composition. For example, the mineral acid can be present in an amount greater than about 3%, greater than about 4%, greater than about 5%, greater than about 6%, greater than about 7%, greater than about 8%, or greater than about 9% based on the weight of the composition.

The amount of negative ions released from the composition can be increased by including one or more of tourmaline, germanium, zeolite powder, or hematite. Each of these components can optionally be included in an amount of less than about 0.5% based on the weight of the composition. For example, each of the components can optionally be included in an amount less than about 0.49%, less than about 0.48%, less than about 0.47%, less than about 0.46%, less than about 0.45%, less than about 0.44%, less than about 0.43%, less than about 0.42%, less than about 0.41%, less than about 0.4%, less than about 0.39%, less than about 0.38%, less than about 0.37%, less than about 0.36%, less than about 0.35%, less than about 0.34%, less than about 0.33%, less than about 0.32%, less than about 0.31%, less than about 0.3%, less than about 0.29%, less than about 0.28%, less than about 0.27%, less than about 0.26%, less than about 0.25%, less than about 0.24%, less than about 0.23%, less than about 0.22%, less than about 0.21%, less than about 0.2%, less than about 0.19%, less than about 0.18%, less than about 0.17%, less than about 0.16%, less than about 0.15%, less than about 0.14%, less than about 0.13%, less than about 0.12%, less than about 0.11%, less than about 0.1%, less than about 0.09%, less than about 0.08%, less than about 0.07%, less than about 0.06%, or less than about 0.05% based on the weight of the composition.

The components described above for use in the methods described herein do not necessarily require further chemical modification prior to use. Further, the components obtained from commercial sources can be used in the methods described herein without further purification. As such, each component can be greater than about 60% pure, greater than about 65% pure, greater than about 70% pure, greater than about 75% pure, greater than about 80% pure, greater than about 85% pure, greater than about 90% pure, or greater than about 95% pure. In some examples, each component can be greater than about 98% pure, greater than about 98.1% pure, greater than about 98.2% pure, greater than about 98.3% pure, greater than about 98.4% pure, greater than about 98.5% pure, greater than about 98.6% pure, greater than about 98.7% pure, greater than about 98.8% pure, greater than about 98.9% pure, greater than about 99% pure, greater than about 99.1% pure, greater than about 99.2% pure, greater than about 99.3% pure, greater than about 99.4% pure, greater than about 99.5% pure, greater than about 99.6% pure, greater than about 99.7% pure, greater than about 99.8% pure, or greater than about 99.9% pure.

In a specific embodiment, the composition includes silicon dioxide in an amount of 23% to 26% based on the weight of the composition; gadolinium in an amount of 0.1% to 0.3% based on the weight of the composition; aluminum oxide in an amount of 1.6% to 1.9% based on the weight of the composition; calcium oxide in an amount of 0.1% to 0.5% based on the weight of the composition; iron oxide in an amount of 0.1% to 0.5% based on the weight of the composition; sodium oxide in an amount of 0.01% to 0.1% based on the weight of the composition; titanium oxide in an amount of 0.2% to 0.4% based on the weight of the composition; zirconium dioxide in an amount of 50% to 60% based on the weight of the composition; phosphoric acid in an amount of 6% to 8% based on the weight of the composition; cerium in an amount of 0.1% to 1% based on the weight of the composition; and sulfur dioxide in an amount of 0.01% to 0.1% based on the weight of the composition. In some examples, the composition further includes neodymium in an amount of 1% to 2% based on the weight of the composition; and praseodymium in an amount of 1% to 2% based on the weight of the composition. In some examples, the composition further includes zeolite powder in an amount of 1% to 2% based on the weight of the composition; and tourmaline in an amount of 1% to 2% based on the weight of the composition. Further examples of the specific compositions are provided in Examples 1 and 2.

As described above, the compositions describe herein can release negative ions. In some examples, the compositions can release up to about 1700 ions per cc, as tested using an AIR Ion Counter for Ore COM-3010PRO (Kintax Trading Co., Ltd; Taiwan). For example, the compositions described herein can release greater than about 50 ions per cc, greater than about 100 ions per cc, greater than about 150 ions per cc, greater than about 200 ions per cc, greater than about 250 ions per cc, greater than about 300 ions per cc, greater than about 350 ions per cc, greater than about 400 ions per cc, greater than about 450 ions per cc, greater than about 500 ions per cc, greater than about 550 ions per cc, greater than about 600 ions per cc, greater than about 650 ions per cc, greater than about 700 ions per cc, greater than about 750 ions per cc, greater than about 800 ions per cc, greater than about 850 ions per cc, greater than about 900 ions per cc, greater than about 950 ions per cc, greater than about 1000 ions per cc, greater than about 1050 ions per cc, greater than about 1100 ions per cc, greater than about 1150 ions per cc, greater than about 1200 ions per cc, greater than about 1250 ions per cc, greater than about 1300 ions per cc, greater than about 1350 ions per cc, greater than about 1400 ions per cc, greater than about 1450 ions per cc, greater than about 1500 ions per cc, greater than about 1550 ions per cc, greater than about 1600 ions per cc, or greater than about 1650 ions per cc.

The compositions as described herein can be prepared by mixing silicon dioxide, gadolinium, and optionally one or more of the additional components as described herein. The resulting composition can be a fine, white or gray powder with a particle size in the range of about 200 to about 400 mesh. For example, the particle size of the composition can be about 210 mesh, about 220 mesh, about 230 mesh, about 240 mesh, about 250 mesh, about 260 mesh, about 270 mesh, about 280 mesh, about 290 mesh, about 300 mesh, about 310 mesh, about 320 mesh, about 330 mesh, about 340 mesh, about 350 mesh, about 360 mesh, about 370 mesh, about 380 mesh, or about 390 mesh.

The compositions described herein can be used as components in products, such as in wristbands, insoles, clothing items, and hats. Examples of suitable clothing items include shirts, pants, socks, and underwear. In some examples, the products can further include adhesive materials. These adhesive materials can include one or more additional negative ion releasing components as described herein (e.g., tourmaline). The additional negative ion releasing components, excluding the negative ion releasing components present in the composition, can be present in the adhesive composition in an amount from about 0.001% to about 2% based on the weight of the product. For example, the additional negative ion releasing component can be present in an amount less than about 2%, less than about 1.9%, less than about 1.8%, less than about 1.7%, less than about 1.6%, less than about 1.5%, less than about 1.4%, less than about 1.3%, less than about 1.2%, less than about 1.1%, less than about 1%, less than about 0.9%, less than about 0.8%, less than about 0.7%, less than about 0.6%, less than about 0.5%, less than 0.4%, less than about 0.3%, less than about 0.2%, less than about 0.1%, or less than about 0.05% based on the weight of the product.

The compositions described herein can be delivered to a subject by applying the composition to a product and contacting the subject with the product. Negative ions are released upon contacting the subject with the composition-containing product. Examples of suitable products include, but are not limited to, fibrous textiles (e.g., cotton, linen, jute, wool, silk, asbestos, and viscose) and chemical fibers (e.g., polyester, nylon, acetate, polypropylene, and rayon). Textiles can be woven, knitted, or machine-knitted, or be present as a composite material (non-woven textile). Non-woven materials can be obtained from, for example, viscose, cotton, cellulose, jute, hemp, sisal, silk, wool, polypropylene, polyester, polyethylene terephthalate (PET), aramide, nylon, polyvinyl derivatives, polyurethanes, polylactide, polyhydroxyalkanoate, cellulose esters and/or polyethylene, and also mineral fibers, such as glass fibers or carbon fibers. Examples of suitable fabrics for use as products also include blends of dual or multiple fibers such as, but not limited to, polyester/elastane blends, polyamids, polyamide/elastane blends, cotton/polyester/elastane blends, polyacrylonitriles, acetates, modal, lyocell and linens. In some examples, the product is comprised primarily of polyester. For example, the product can be comprised of 60% or greater, 70% or greater, 80% or greater, 90% or greater, or 100% polyester by weight of the product.

Examples of suitable products onto or into which the compositions described herein can be applied include clothing items and accessories, personal care items, bandages, beddings, carpets/rugs, paints/decorations, leather, sporting articles, protective gears, or the like. In some examples, the product is a clothing item (e.g., a shirt) or a wristband.

The compositions described herein can be applied onto a finished article of manufacture, such as an item of clothing, using transfer printing, screen-printing, ink-jet printing, pressing rollers, or other techniques as known to those of skill in the art. Alternatively, the compositions can be applied to the item prior to the production (e.g., during the fabrication of the item). For example, the compositions described herein can be coated onto a thread for sewing or weaving (e.g., a polyester, cotton, silk or nylon thread) entering into the fabrication of the clothing item.

In some embodiments, the compositions including the negative ions are printed in a prescribed design on the product. For example, the compositions can be printed in prescribed designs on one or more areas of a shirt. In other embodiments, the compositions can be shaped and then attached (e.g., by sewing) to one or more areas of the shirt. Suitable areas of the shirt for printing the prescribed design and/or sewing the shaped compositions include one or both the sleeve cuffs and on the neckband of the shirt. In some examples, the shirt is a 100% polyester shirt. By wearing this shirt, negative ions are delivered to the subject through the skin of the subject. The amount of composition to be applied to the product, the distribution of the composition on the product, and the period of exposure to the composition depend on the desired effect and will thus be apparent to one of skill in the art.

The following non-limiting examples are provided to more fully illustrate some particular embodiments. Parts and percentages are provided on a per weight basis except as otherwise indicated.

### EXAMPLES

### Example 1

### Composition Formulation

Exemplary ingredients for a negative ion-releasing silica composition as described herein are shown in Table 1.

TABLE 1

| Ingredients | Parts by weight of composition |
|---|---|
| Silicon Dioxide | 25.6 |
| Gadolinium | 0.28 |
| Alumina | 1.82 |
| Calcium Oxide | 0.19 |
| Magnesium Oxide | 0.28 |
| Iron Oxide | 0.39 |
| Sodium Oxide | 0.05 |
| Titanium Oxide | 0.35 |
| Zirconia | 54.4 |
| Phosphoric Acid | 6.91 |
| Cerium | 0.55 |
| Sulfur Dioxide | 0.07 |
| Neodymium | 1.79 |
| Praseodymium | 1.32 |
| Additional Component(s) | 0-0.5 |

In the above formulation, the additional component is selected from tourmaline crushed crystals, germanium, zeolite powder, hematite, and mixtures of these. The components are mixed together to form a fine white powder (325 mesh).

### Example 2

### Composition Formulation

Exemplary ingredients for a negative ion-releasing silica composition as described herein are shown in Table 2.

TABLE 2

| Ingredients | Parts by weight of composition |
|---|---|
| Silicon Dioxide | 25.6 |
| Gadolinium | 0.28 |
| Alumina | 1.82 |
| Calcium Oxide | 0.19 |
| Magnesium Oxide | 0.28 |
| Iron Oxide | 0.39 |
| Sodium Oxide | 0.05 |
| Titanium Oxide | 0.35 |
| Zirconia | 54.4 |
| Phosphoric Acid | 6.91 |
| Cerium | 0.55 |
| Sulfur Dioxide | 0.07 |
| Zeolite Powder | 1.79 |
| Tourmaline Crushed Crystals | 1.32 |
| Additional Component | 0-0.5 |

In the above formulation, the additional component is selected from germanium, hematite, and mixtures of these. The components are mixed together to form a fine white powder (325 mesh).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative composition materials and method steps disclosed herein are specifically described, other combinations of the composition materials and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed.

What is claimed is:

1. A composition, comprising:
silicon dioxide; and
gadolinium.

2. The composition of claim 1, wherein the silicon dioxide is present in an amount of 20% to 30% based on the weight of the composition.

3. The composition of claim 1, wherein the gadolinium is present in an amount of 0.1% to 0.5% based on the weight of the composition.

4. The composition of claim 1, further comprising a lanthanide.

5. The composition of claim 4, wherein the lanthanide is selected from the group consisting of neodymium, praseodymium, cerium, or a mixture of these.

6. The composition of claim 4, wherein the lanthanide is present in an amount of 2% to 5% based on the weight of the composition.

7. The composition of claim 1, further comprising tourmaline, germanium, zeolite powder, hematite, or a mixture of these.

8. The composition of claim 1, further comprising an additional oxide.

9. The composition of claim 8, wherein the additional oxide is selected from the group consisting of aluminum oxide, calcium oxide, iron oxide, magnesium oxide, sodium oxide, sulfur dioxide, titanium dioxide, zirconium dioxide, or a mixture of these.

10. The composition of claim 8, wherein the additional oxide is present in an amount of 40% to 65% based on the weight of the composition.

11. The composition of claim 1, further comprising a mineral acid.

12. The composition of claim 11, wherein the mineral acid is phosphoric acid.

13. A composition, comprising:
silicon dioxide in an amount of 23% to 26% based on the weight of the composition;
gadolinium in an amount of 0.1% to 0.3% based on the weight of the composition;
aluminum oxide in an amount of 1.6% to 1.9% based on the weight of the composition;
calcium oxide in an amount of 0.1% to 0.5% based on the weight of the composition;
iron oxide in an amount of 0.1% to 0.5% based on the weight of the composition;
sodium oxide in an amount of 0.01% to 0.1% based on the weight of the composition;
titanium oxide in an amount of 0.2% to 0.4% based on the weight of the composition;
zirconium dioxide in an amount of 50% to 60% based on the weight of the composition;
phosphoric acid in an amount of 6% to 8% based on the weight of the composition;
cerium in an amount of 0.1% to 1% based on the weight of the composition; and
sulfur dioxide in an amount of 0.01% to 0.1% based on the weight of the composition.

14. The composition of claim 13, further comprising neodymium in an amount of 1% to 2% based on the weight of the composition; and praseodymium in an amount of 1% to 2% based on the weight of the composition.

15. The composition of claim 13, further comprising zeolite powder in an amount of 1% to 2% based on the weight of the composition; and tourmaline in an amount of 1% to 2% based on the weight of the composition.

16. A product comprising the composition of claim 1, wherein the product is a wristband, an insole, a clothing item, or a hat.

17. The product of claim 16, further comprising an adhesive.

18. The product of claim 17, wherein the adhesive comprises tourmaline.

19. The product of claim 16, wherein the clothing item is a shirt.
